SEQUENCE LISTING (13 sequences)

SEQ ID NO: 1 - DNA, 69 bp, Artificial Sequence
  Description of Artificial Sequence Synthetic primer
tctaaatccatatcctcataagcagcaatcaattctatctatactttaaacagctgaagcttcgtacgc

SEQ ID NO: 2 - DNA, 72 bp, Artificial Sequence
  Description of Artificial Sequence Synthetic primer
attaaattttacttttattacatacaactttttaaactaatatacacattgcataggccactagtggatctg

SEQ ID NO: 3 - DNA, 69 bp, Artificial Sequence
  Description of Artificial Sequence Synthetic primer
gcacaatatttcaagctataccaagcatacaatcaactatctcatatacacagctgaagcttcgtacgc

SEQ ID NO: 4 - DNA, 72 bp, Artificial Sequence
  Description of Artificial Sequence Synthetic primer
ttttttataacttatttaataataaaaatcataaatcataagaaattcgcgcataggccactagtggatctg

SEQ ID NO: 5 - DNA, 21 bp, Artificial Sequence
  Description of Artificial Sequence Synthetic primer
cgcaagtcctgtttctatgcc

SEQ ID NO: 6 - DNA, 23 bp, Artificial Sequence
  Description of Artificial Sequence Synthetic primer
ttccaagtccaagattgaagctg

SEQ ID NO: 7 - DNA, 20 bp, Artificial Sequence
  Description of Artificial Sequence Synthetic primer
tctctctcccccgttgttgt

SEQ ID NO: 8 - DNA, 20 bp, Artificial Sequence
  Description of Artificial Sequence Synthetic primer
ctcaggtaaggggctagtag

SEQ ID NO: 9 - DNA, 18 bp, Artificial Sequence
  Description of Artificial Sequence Synthetic primer
ggatgtatgggctaaatg

SEQ ID NO: 10 - DNA, 18 bp, Artificial Sequence
  Description of Artificial Sequence Synthetic primer
cctcgacatcatctgccc

SEQ ID NO: 11 - DNA, 23 bp, Artificial Sequence
  Description of Artificial Sequence Synthetic primer
agtcacatcaagatcgtttatgg

SEQ ID NO: 12 - DNA, 23 bp, Artificial Sequence
  Description of Artificial Sequence Synthetic primer
gcacggaatatgggactacttcg

SEQ ID NO: 13 - DNA, 23 bp, Artificial Sequence
  Description of Artificial Sequence Synthetic primer
actccacttcaagtaagagtttg

(19) United States
(12) Patent Application Publication
(10) Pub. No.: US 2011/0124075 A1
(43) Pub. Date: May 26, 2011

(54) CONSTRUCTION OF GENETICALLY TRACTABLE INDUSTRIAL YEAST STRAINS

(75) Inventors: Alexander Amerik, Norwalk, CT (US); Steven A. Henck, Woodbridge, CT (US)

(73) Assignee: Arbor Fuel Inc., Woodbridge, CT (US)

(21) Appl. No.: 12/625,275

(22) Filed: Nov. 24, 2009

(51) Int. Cl. C12P 1/02; C12N 1/18; C12N 1/19

(52) U.S. Cl. 435/171; 435/255.2; 435/254.21

(57) ABSTRACT

Embodiments of the present invention include genetically tractable industrial yeast strains and methods for their construction. In certain preferred embodiments, the genetically tractable industrial yeast strain is a Saccharomyces cerevisiae strain, such as a derivative of the K1-V1116 wine yeast strain.

### FIELD OF THE INVENTION

This invention relates to recombinant microorganisms, for example the yeast Saccharomyces cerevisiae (S. cerevisiae), as well as methods for their construction and manufacture, that are genetically tractable and suitable for industrial processes, for example the conversion of cellulosic materials to ethanol or butanol.

### BACKGROUND OF THE INVENTION

Traditional application of industrial yeast includes the production of commercially and societally important products, such as bread, wine and other alcoholic beverages, generally as a result of fermentation processes. The most commonly used, including for ethanol production, industrial yeast is S. cerevisiae. As it has been used industrially for thousands of years, S. cerevisiae has been referred to as “the first domesticated microorganism” (Borneman et al., 2008). Accordingly, industrial yeast strains have been subjected to selection pressures and isolated for improved industrial performance, such as fermentative ability under a variety of stressful environmental conditions (See id.). Other features commonly found in industrial yeast are efficient sugar utilization, high ethanol tolerance and production, high fermentation rate and, finally, genetic stability, a factor that provides the reproducibility of the characteristics of final products (Kelsall and Lyons, 2003).

Another set of yeast strains, e.g., strains of S. cerevisiae, have been isolated for their use in non-industrial applications, for example, for use in basic biological research. As a result, these laboratory yeast strains have been subjected to different selection pressures than industrial yeast strains. For example, laboratory strains have been selected to be able to grow well in nutrient rich media. Furthermore, laboratory yeast strains generally behave regularly in crosses and mating and carry convenient genetic markers widely used in academic research. As a result, molecular genetics of laboratory yeast strains is very well developed. However, laboratory strains frequently display reduced growth rate and require expensive growth media. Therefore, they are not suitable for industrial applications. In one study comparing the sequences of an industrial S. cerevisiae strain genome with a laboratory S. cerevisiae strain genome, significant differences were found between the two strains (Borneman et al., 2008).

Industrial yeast strains are still poorly characterized genetically. They are generally considered to be organisms with a great deal of gene heterogeneity and even unknown chromosomal constitution. Engineering genetically modified yeast suitable for industrial applications requires molecular genetic analysis that necessarily includes sporulation of diploid cells and investigation of segregation of genetic markers. Unfortunately, industrial strains sporulate poorly and rarely form 4-spore asci. Furthermore, germination efficiency (spore viability) is very low. For example, Johnston et al. (2000) analyzed sporulation and spore viability of 13 widely used wine yeast strains. Among them, only one strain, S6U, showed 95% spore viability appropriate for genetic manipulation. However, this strain sporulated with low efficiency.

The K1-V1116 strain of S. cerevisiae is widely used in the wine industry. It has several advantages over other yeast strains including high tolerance to alcohol (up to 18%), particularly short lag phase, very large range of fermentation temperature (10 to 35° C.) and low average requirement for assimilable nitrogen. This strain is also resistant to difficult fermentation conditions such as low turbidity, low temperature and low fatty acid content (Lalvin ICV-K1 (V1116) product literature; www.lalvinyeast.com). Importantly, K1-V1116 yeast can be adapted for better bioethanol production. It was shown that genetically modified K1-V1116 derivative achieved about 18% improvement in its glucose-to-ethanol conversion efficiency compared to the respective parent strain (Ooi and Lankford, 2009).

Applicants' data and data from others (Johnston et al., 2000) show that the K1-V1116 yeast sporulate reasonably well and that asci contained four spores. However, only 50% of spores were viable, suggesting that wild-type K1-V1116 yeast has a lethal recessive mutation. Furthermore, surviving spores form colonies of different size indicating that other mutations or their combination may also affect growth rate. These obstacles severely impede genetic analysis, which is critical for mutant construction, engineering metabolic pathways and other manipulations.

Thus, there is a need in the art for genetically tractable industrial yeast strains and methods for their construction.

### SUMMARY OF THE INVENTION

Described herein are genetically tractable industrial yeast strains and methods for their construction. In certain preferred embodiments, the genetically tractable industrial yeast strain is a Saccharomyces cerevisiae strain. This simple eukaryote is a robust tool for industrial fermentation processes and is commonly used for ethanol production. S. cerevisiae has many attractive features that make it one of the major hosts for metabolic engineering in science and industry. Not only can S. cerevisiae cells be genetically transformed with ease, but they also have rapid growth rates, and new genetic information can be introduced via chromosomal integration or via the introduction of stable plasmid DNA. Genetic analysis is an obligatory part of research aimed at metabolic engineering of microorganisms. Molecular genetics of the laboratory yeast strains is very well developed. It provides a unique opportunity, at a level not yet available in almost any other prokaryotic or eukaryotic organisms, of both genetic manipulations and detailed biochemical analysis. However, laboratory strains frequently display reduced growth rate and require expensive growth media. Therefore, they are not suitable for industrial applications. By contrast, industrial strains are robust and can grow without expensive supplements. Unfortunately, several features of industrial yeast including polyploidy, low sporulation efficiency, germination defects, etc. severely impair molecular genetic characterization of these strains.

Applicants efforts to construct genetically tractable industrial yeast strains are described herein. More specifically described herein is the construction of K1-V1116 (Lallemand, Montreal, QC, Canada) yeast derivatives suitable for detailed genetic manipulation and analysis. Specifically, Applicants dramatically improved sporulation efficiency of these cells, knocked out the pathway responsible for the mating type switch and constructed stable haploids of both mating type. In particular, Applicants first performed several rounds of sporulation/dissection of the K1-V1116 derivatives resulted from viable spores, which led to isolation of two strains that had sporulation efficiency close to 100%. Applicants also deleted the HO gene responsible for mating type switch in these strains and isolated stable heterothallic haploids of both a and α mating types. Finally, Applicants constructed heterothallic diploid strains. Remarkably, the foregoing genetic manipulations did not affect the growth or fermentation properties of this yeast essential for industrial application. Thus, Applicants engineered yeast strains (e.g., the yeast having ATCC Accession No. PTA-10443) that possess the robustness of industrial yeast and that provide the tools for molecular genetic analysis.

In another aspect, methods of producing, and optionally recovering, a fermentation product are provided that include fermenting a carbon substrate with a yeast strain described herein under conditions where the fermentation product is produced. For example, a yeast strain according to embodiments of the present invention is provided and used to convert cellulosic materials to butanol as described in International Application Publication No. WO2009/055072, or in U.S. application Ser. No. 12/589,715, filed on Oct. 26, 2009, each of which is hereby incorporated by reference in its entirety. In another example, a yeast strain according to embodiments of the present invention is provided and used to convert cellulosic materials to ethanol as described in U.S. Application Publication No. US 2009-0246844 or in International Application No. PCT/US09/05836, filed on Oct. 26, 2009, each of which is hereby incorporated by reference in its entirety.

In yet another aspect, methods of manufacturing a yeast strain are provided that include culturing a yeast strain described herein in a medium under conditions where the yeast strain grows.

It is contemplated that whenever appropriate, any embodiment of the present invention can be combined with one or more other embodiments of the present invention, even though the embodiments are described under different aspects of the present invention.

### BRIEF DESCRIPTION OF THE FIGURES

The invention can be more fully understood from the following detailed description, figure, and the accompanying sequence descriptions, which form a part of this application.

FIG. 1 shows the spore viability and growth of the K1-V1116 wine yeast and its derivatives. (A) Defect in spore viability and growth of K1-V1116. Arrow indicates the primary colonies that were sporulated and dissected. (B) The AFY150 strain shows 100% spore viability. The AFY157 and AFY159 depicted by arrows were selected for further genetic manipulation.

FIG. 2 depicts a strategy for the construction of genetically tractable industrial yeast strains.

FIG. 3 shows a map of plasmid pUG6 carrying the loxP-kanMX-loxP disruption module and gene disruption using the loxP-kanMX-loxP disruption cassette. For gene disruption experiments, two oligonucleotides were synthesized (Table 2) with their 3′ ends complementary to sequences left and right of the loxP-his5-loxP module on plasmid pUG6 and with their 5′ ends complementary to the 5′ and 3′ flanking regions of the HO gene. Plasmid pUG6 was used as PCR template to generate the disruption cassette.

FIG. 4 shows kanMX marker rescue by expression of Cre recombinase. The heterothallic diploid kanMX⁺ yeast strains were transformed with plasmid pSH65. Transformants were grown in glucose medium and then shifted to galactose medium to induce expression of Cre recombinase. Cre recombinase induced recombination process between two loxP sites that removes the marker gene.

FIG. 5 depicts verification of marker rescue by diagnostic PCR and by ability of yeast cells to grow on G418-containing media. (A) Two verification primers, HOA and HOD (Table 2) were used in a PCR to verify the ho deletion and kanMX rescue. All engineered strains analyzed showed a band of 1.03 kb as expected for deleted HO locus without integrated kanMX gene. By contrast, the wild-type K1-V1116 cells showed a band of 2.7 kb corresponding to the intact HO locus. (B) After rescue of the kanMX marker by Cre recombinase the AFY201-206 strains lost the ability to grow on G418-containing media. The AFY191 and AFY192 strains that were not treated by Cre recombinase grew in the presence of G418 concentrations of 200 μg/mL.

FIG. 6 shows determination of the mating type by PCR. Three oligonucleotide primers, MATtest, MATa and MATalpha, were used in a single PCR reaction. DNA at MATa locus generated 0.54 kb product, whereas DNA at MATα locus generated 0.40 kb product. Diploid cells showed both bands. (A) PCR mating type analysis of dissection products of heterozygous HO/ho::kanMX diploids. MATa and MATα bands are depicted by arrows. (B) The AFY201-203 heterothallic diploids generate stable haploid derivatives.

FIG. 7 shows the fermentation of ethanol from 2% glucose under anaerobic conditions using adh1 mutants AFY229 and AFY235 is substantially lower than with their parental strains AFY213 and AFY214.

### DETAILED DESCRIPTION OF THE INVENTION

The highly developed genetics of the yeast Saccharomyces cerevisiae is usually one of the key features to choose this simple eukaryote as an organism for recombinant DNA work. A large group of related laboratory yeast strains that behave regularly in crosses and mating, and carry convenient genetic markers are widely used in academic research. However, these strains are ill suited for traditional practical application such as bread-making and fermentation. On the other hand, industrial yeast strains suffer from difficulties when it comes to applying various genetic techniques. Applicants' approaches to dealing with these difficulties are described below.

Unless defined otherwise, all technical and scientific terms used herein have the same meaning as commonly understood by one of ordinary skill in the art to which this invention belongs. In the case of conflict, the present specification will control. The following definitions and abbreviations are to be used for the interpretation of the claims and the specification.

The term “ethanol biosynthetic pathway” refers to a microbial pathway to produce ethanol.

The term “carbon substrate” refers to a carbon source capable of being metabolized by yeast strains of the present invention, and particularly carbon sources selected from the group consisting of monosaccharides, oligosaccharides, polysaccharides, or mixtures thereof.

The term “fermentation product” refers to the final or intermediate product(s) of fermentation by yeast strains of the present invention. Fermentation products can include, but are not limited to, succinate, lactate, acetate, ethanol, formate, carbon dioxide, hydrogen gas, 1,3-propanediol, 2,3-butanediol, acetoin, propionate, butyrate, butanol, acetone, singly or mixtures thereof. Fermentation products include both oxidized and reduced products of fermentation.

The term “gene” refers to a nucleic acid fragment that is capable of being expressed as a specific protein or RNA, optionally including regulatory sequences preceding (5′ non-coding sequences) and following (3′ non-coding sequences) the coding sequence. “Native gene” refers to a gene as naturally found in a host organism with its own regulatory sequences. “Chimeric gene” refers to any gene that is not a native gene, comprising regulatory and coding sequences that are not found together in the host organism. Accordingly, a chimeric gene may comprise regulatory sequences and coding sequences that are derived from different sources, or regulatory sequences and coding sequences derived from the same source, but arranged in a manner different than that found in that source. “Endogenous gene” refers to a native gene in its natural location in the genome of an organism. A “foreign gene” or “heterologous gene” refers to a gene not normally found in the host organism, but that is introduced into the host organism. Foreign genes can comprise native genes inserted into a non-native organism, or chimeric genes. It is also understood, that foreign genes encompass genes whose coding sequence has been modified to enhance its expression in a particular host, for example, codons can be substituted to reflect the preferred codon usage of the host.

As used herein the term “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structures.

The term “promoter” refers to a DNA sequence capable of controlling the expression of a coding sequence or functional RNA (e.g., rRNA). A coding sequence is located downstream of a promoter sequence. Promoters may be derived in their entirety from the promoter of a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from nucleic acid fragments. Expression also refers to translation of mRNA into a polypeptide.

As used herein, the term “transformation” refers to the insertion of an exogenous nucleic acid into a cell, irrespective of the method used for the insertion, for example, lipofection, transduction, infection or electroporation. The exogenous nucleic acid can be maintained as a non-integrated vector, for example, a plasmid, or alternatively, can be integrated into the cell's genome. Host organisms containing the transformed nucleic acid fragments are referred to as “transgenic” or “recombinant” or “transformed” organisms.

The terms “plasmid”, “vector” and “cassette” refer to an extrachromosomal element often carrying genes which are not part of the central metabolism of the cell, and usually in the form of circular double-stranded DNA fragments. Such elements can be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation vector” refers to a vector or linear DNA fragment containing a foreign gene and having elements in addition to the foreign gene that facilitates transformation of a particular host cell. “Expression vector” refers to a vector containing a foreign gene and having elements in addition to the foreign gene that allow for enhanced expression of that gene in a foreign host.

The terms “inactivate” or “inactivation” as used herein with reference to a biologically active molecule, such as an enzyme, indicates any modification in the genome and/or proteome of a microorganism that prevents or reduces the biological activity of the biologically active molecule in the microorganism. Exemplary inactivations, include but are not limited to, modifications that results in the conversion of the molecule from a biologically active form to a biologically inactive form and from a biologically active form to a biologically less or reduced active form, and any modifications that result in a total or partial deletion of the biologically active molecule. For example, inactivation of a biologically active molecule can be performed by deleting or mutating a gene encoding the biologically active molecule in the microorganism, by deleting or mutating a gene encoding an enzyme involved in the pathway for the synthesis of the biologically active molecule in the microorganism. In particular, in some embodiments inactivation of a biologically active molecule such as an enzyme can be performed by deleting from the genome of the recombinant microorganism one or more endogenous genes encoding for the enzyme.

As used herein, “deleting genes” means that a gene is deleted or otherwise mutated to inactivate the gene. Deletions can be of coding sequences or regulatory sequences provided they do not tend to revert and provided they inactivate the gene product (or gene products as the case may be).

As used herein, the term “industrial yeast strain” refers to a yeast strain that is suitable for use for the industrial fermentation or the production of chemicals (e.g., for the production of biofuels, bread or alcoholic beverages, such as wine or beer). Industrial yeast strains include, but are not limited to, strains used for commercial and amateur winemaking, beer brewing and bread making. An industrial yeast strain will have one or more, preferably all, of the following characteristics: intrinsic tolerance to the ethanol, high temperature tolerance, and high growth rate. For example, in some embodiments, the industrial yeast strain will tolerate ethanol concentrations of greater than about 15%, greater than about 18%, greater than about 20% or greater than about 22%. In some embodiments, the industrial yeast strain will tolerate temperatures greater than 37° C. In some embodiments, the industrial yeast strain will tolerate temperatures of at least about 34° C., at least about 35° C., at least about 36° C., or at least about 37° C. In some embodiments, the industrial yeast strain will have a growth rate, such that the doubling time of the number of yeast cells is less than about 120 minutes, for example, between about 90 minutes and about 120 minutes, or about 100 minutes, or about 90 minutes, or less than about 90 minutes.

Standard molecular biology techniques used herein are well known in the art and are described by Sambrook J, Fritsch E F, Maniatis T. 1989. Molecular Cloning: A Laboratory Manual. Cold Spring Harbor Laboratory Press: Cold Spring Harbor, N.Y. Techniques for manipulation of S. cerevisiae used herein are well known in the art and are described in Methods in Yeast Genetics. 2005. Cold Spring Harbor Laboratory Press: Cold Spring Harbor, N.Y., and in Guthrie C, Fink G R, (Eds.). 2002. Methods in Enzymology, Volume 351, Guide to Yeast Genetics and Molecular and Cell Biology (Part C), Elsevier Academic Press, San Diego, Calif.

### Sporulation of the Industrial Yeast Strains

Reduction of chromosome numbers by sporulation is a prerequisite for genetic analysis, cross-breeding and mutation-breeding. Many industrial strains, however, do not sporulate well when subjected to conditions standard for laboratory strains. To increase sporulation efficiency of the industrial yeast strains, several approaches have been suggested. These include using sporulation media containing a small amount of glucose and yeast extract to allow a gradual shift from mitotic growth to sporulation, sporulation at lower temperature, addition of certain elements (e.g., zinc, 25 ppm.) and compounds (e.g., S-lactoylglutothione, 5 mM) that stimulate sporulation (Kielland-Brandt, 1994). However, these techniques lead to only partial improvement of sporulation efficiency of the industrial yeast strains. In addition to the sporulation defect, industrial yeast strains display germination defects. The percentage of spores that can survive (germinate) is frequently very low and complete tetrads are not generated after sporulation and dissection.

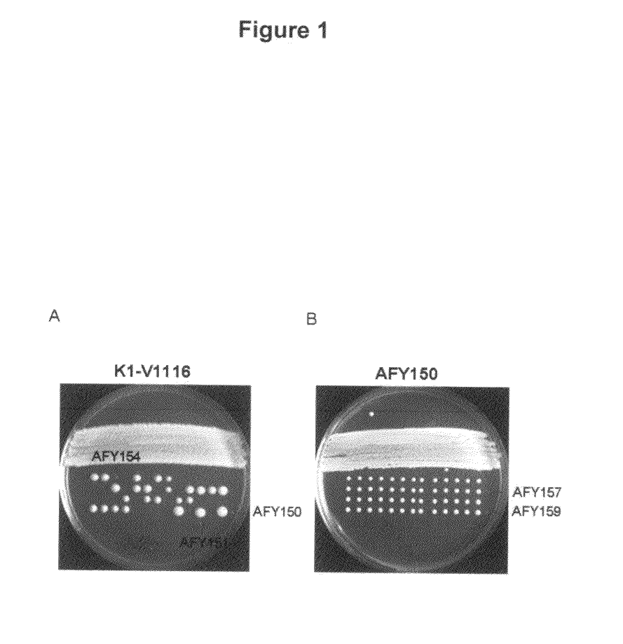

In order to identify industrial yeast suitable for genetic manipulation, Applicants investigated sporulation efficiency and spore viability of two well known distillery strains—ATCC 4124 (American Type Culture Collection, Manassas, Va.) and SuperStart (White Labs, Boulder, Colo.) and two wine strains—K1-V1116 and EC-1118 (both from Lalvin, Canada). Surprisingly, sporulation efficiency of these strains was relatively high and a vast majority of asci contained four spores. However, spore viability was low. The survival rate was 8%, 9%, 29% and 48% for ATCC 4124, SuperStart, EC-1118 and K1-V1116, respectively. Interestingly, the ATCC 4124, SuperStart and EC-1118 derivatives displayed severe growth defects suggesting that these strains missed genetic information essential for maximum rate of growth. As expected, derivatives of these three strains were not able to produce complete tetrads following sporulation. Applicants research focused on K1-V1116 for two reasons. First, as noted, these cells showed maximum spore viability. Second, segregation of viability was 2:2 for 12 out of 13 spores (FIG. 1A). This observation suggested that the K1-V1116 diploid yeast might have a recessive lethal mutation that could be separated from the wild-type allele by sporulation and subsequent tetrad dissection. In addition, the obvious growth defect seen in ˜50% of surviving spores indicated that other mutations or their combination(s) might also affect growth of the K1-V1116 derivatives.

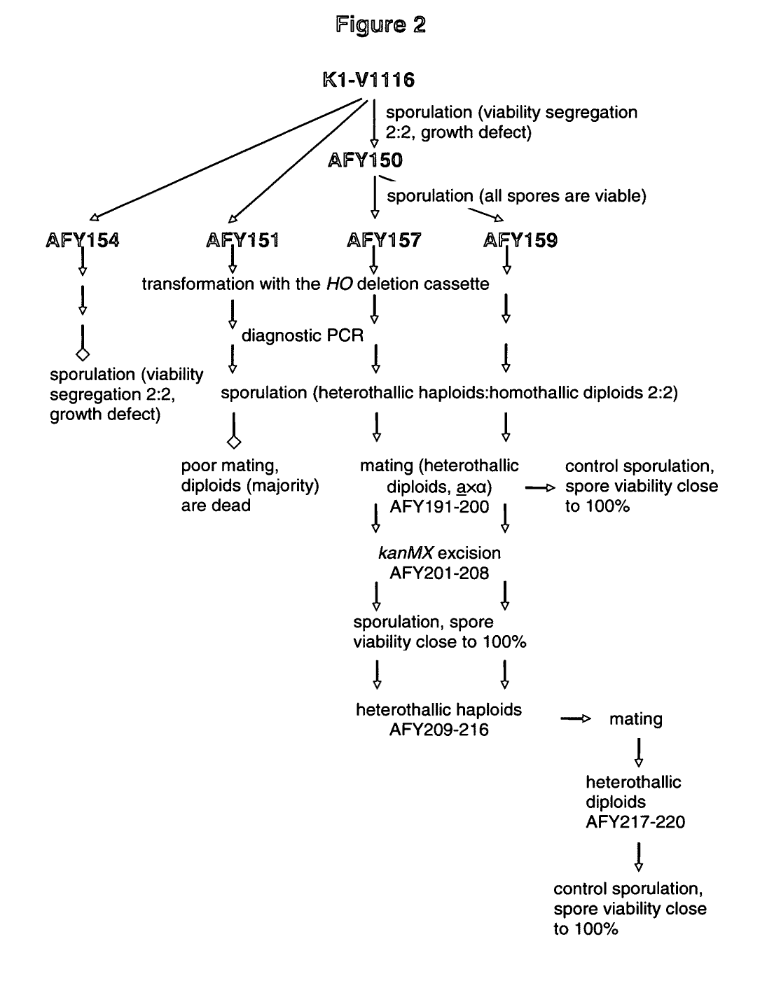

If the above suggestions are correct, dissection products shown in FIG. 1A should produce complete tetrads. Indeed all 52 spores derived from AFY150 were viable. Moreover, cells derived from the germinated spores grew at very similar rates at 30° C. and 37° C. indicating the AFY150 strain did not contain mutations affecting growth (FIG. 1B). Remarkably, AFY150 and its derivatives were different from other tested products of the primary spores. For example, the AFY154 cells produced only 50% viable spores (FIG. 2). Heterothallic haploids derived from another K1-V1116 product, AFY151, showed severe mating defects (FIG. 2). Thus, genetic selection performed by Applicants led to isolation of an industrial yeast strain that sporulated reasonably well and, more importantly, produced ˜100% viable spores. Two AFY150 derivatives, clones 1b (AFY157) and 1d (AFY159) were chosen for further analysis (FIG. 1B).

### Deletion of the HO Gene in the Yeast Genome. Identification of the Heterothallic Haploids of Both, a and α, Mating Types

The yeast Saccharomyces cerevisiae is a diploid organism. After germination of the haploid spore a fraction of the yeast cells spontaneously change mating type and then mate to form diploids. The HO gene encodes an endonuclease responsible for initiating mating-type switching, a gene conversion process where MATa cells change to MATα cells or vice versa. Cell mating type, MATa or MATα is determined by information expressed from the MAT locus. The mating type information is stored in two transcriptionally silenced loci, HMLα and HMRa (Nasmyth, 1982; Herskowitz, 1998). HO initiates switching by recognizing and cleaving a degenerate 24 base-pair site at MAT making a double-stranded break in DNA (Nickoloff et al., 1986). Sequences at MAT are then replaced by copying new sequences from either HML or HMR (Strathern et al., 1982). HO expression and hence, mating-type inter-conversion, occurs exclusively in haploid cells.

Normally industrial yeast strains are diploids or polyploids. However, heterothallic industrial haploids may be useful for many biotechnological applications. For example, two metabolic pathways can be combined in a single cell after mating haploids of opposite mating type. In addition, expression level, and therefore potentially activity of exogenous proteins, can be increased by mating recombinant haploids.

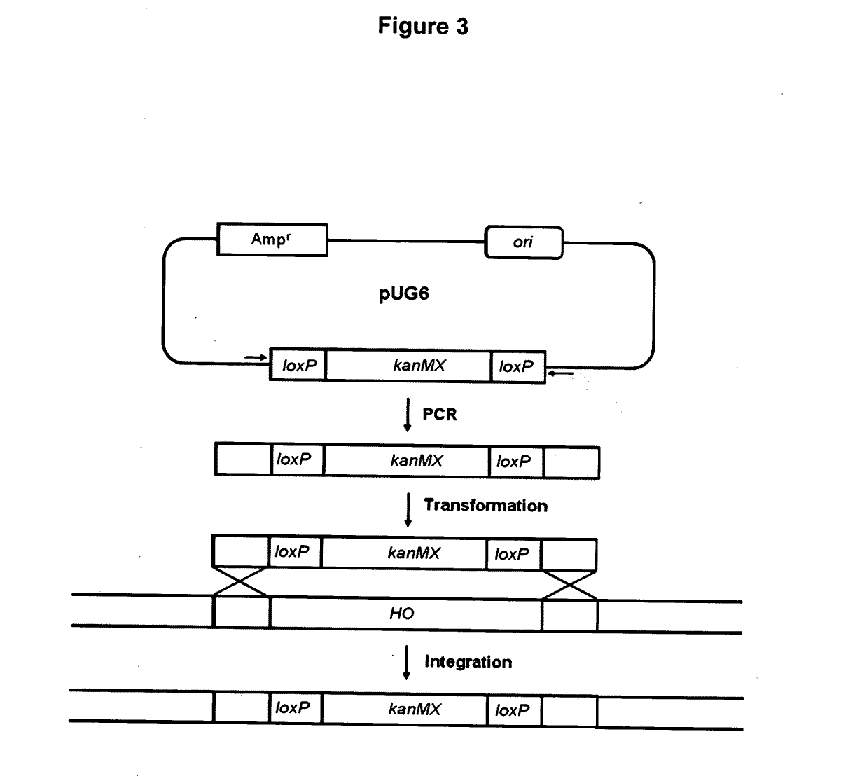

To delete the HO gene, a gene disruption technique was employed. Specifically, two HO-specific oligonucleotide primers were synthesized (Table 2). Each primer contained a 3′ end corresponding to sequences flanking the loxP-kanMX-loxP disruption cassette. The gene-specific 5′ end was identical to sequence either upstream or downstream of the HO ORF (FIG. 3). The 3′ ends allowed amplification of the disruption cassette encoding the kanMX marker flanked by two loxP repeats. The 5′ ends secured precise replacement of the HO gene by amplified DNA. After transformation of the AFY151, AFY154, AFY157 and AFY159 strains with the disruption cassette by electroporation, G418 resistant colonies were verified by diagnostic PCR using HOA/kan-B and HOD/kan-C pairs of primers. The majority of colonies generated PCR fragments of the expected size.

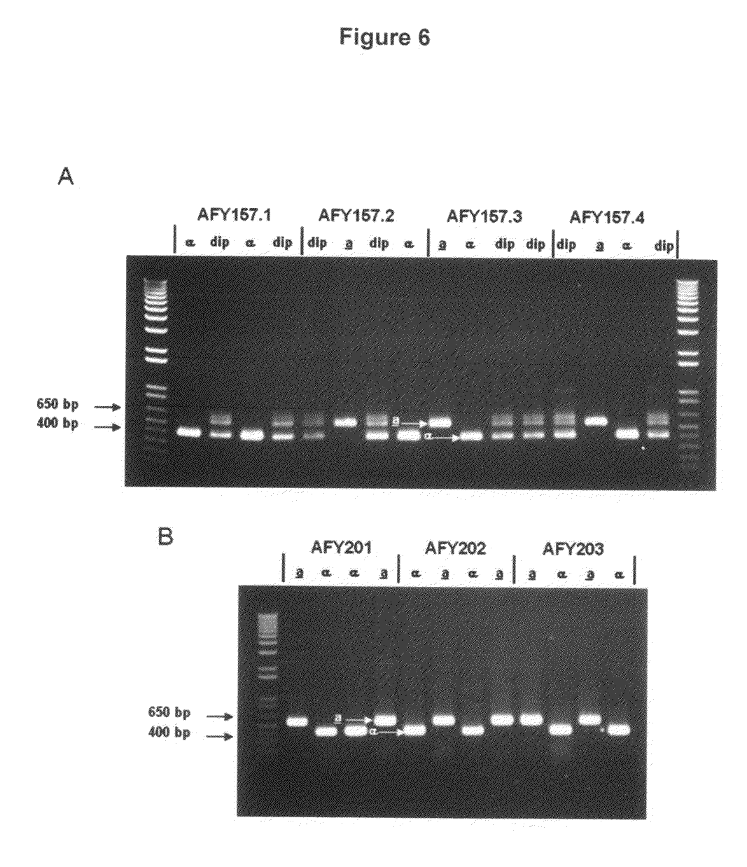

Homologous recombination leads to replacement of only one copy of the gene with the disruption cassette in a diploid genome. Therefore, heterozygous diploids described below were sporulated and resultant tetrads were dissected. The mating type of the resultant products was analyzed by PCR (FIG. 6). As expected, each tetrad contained two mutant and two wild type spores. Products of wild type spores diploidised with a high frequency forming diploid cells. By contrast, mutant spores produced stable heterothallic haploids of both mating types.

As noted, the AFY154 strain did not produce complete tetrads (FIG. 2). Instead, segregation of spore viability was very similar to that seen for the original K1-V1116 cells. This may indicate that the genotype of the K1-V1116 yeast is more complex than suggested. Nevertheless, the AFY151, AFY157 and AFY159 strains demonstrated spore viability close to 100% and cells derived from these spores did not show any phenotypic abnormities (growth defect, heat sensitivity etc.). This is an important observation because surviving spores of many industrial yeast strains, including ATCC and SuperStart, generate strains that are frequently much less robust than the parents.

### Construction of Heterothallic Diploids—Rescue of the kanMX Marker from Diploid Cells

To construct heterothallic diploids, haploid cells of opposite mating type were mated, leading to the isolation of the AFY191-200 strains. These strains were sporulated and tetrads were dissected. In each case spore viability was close to 100%. Strikingly, heterothallic haploids derived from AFY151 displayed severe mating defects. In addition, ˜80% of isolated diploids were nonviable. Therefore, AFY151 derivatives were excluded from further analysis.

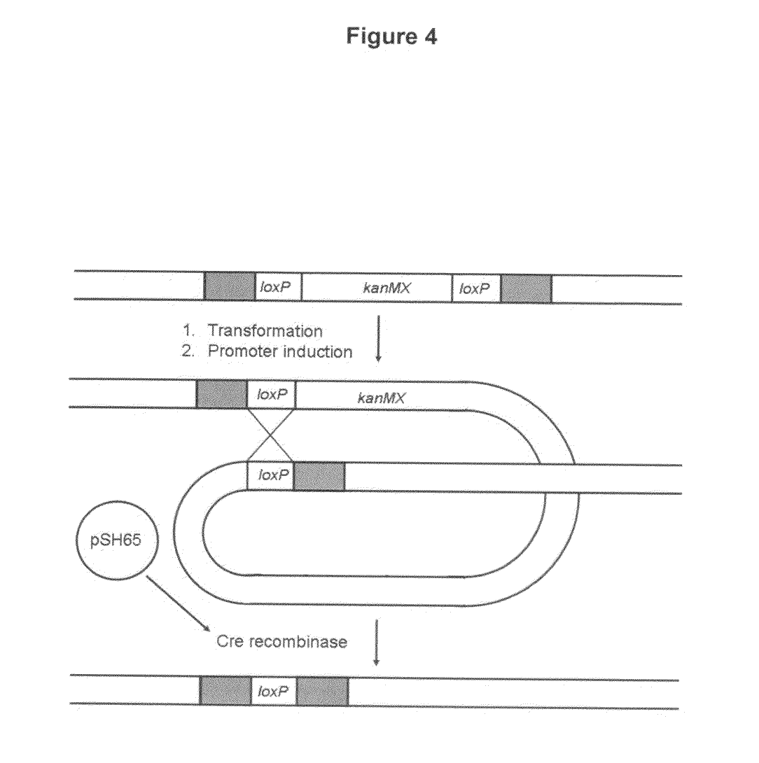

To use the kanMX marker repeatedly for successive gene disruptions in one strain, it is necessary to eliminate the marker from the successfully disrupted gene. Moreover, the foregoing strains have potential problems. First, phenotypes conferred by drug-resistant markers have the potential to change important traits of industrial yeast including alcohol fermentation. Second, the food and drug industries express concern that any exogenous DNA inserted into industrial yeast may create public fear about product safety. Therefore, isolated heterothallic diploids were transformed with the plasmid pSH65, which carries the ble resistant marker gene from transposon Tn5 conferring phleomycin resistance to yeast cells and the Cre recombinase gene under the control of the inducible GAL1 promoter. Expression of the Cre recombinase was induced by shifting cells from YPD (glucose) to YPG (galactose) medium. Growth for only 2 hours in galactose containing medium after transfer from glucose medium was sufficient to remove the kanMX marker gene in 80-90% of the cells, as detected by plating cells on YPD and replica-plating the colonies onto YPD plus G418. Loss of the kanMX marker gene was verified by growth on G418-containing medium and by diagnostic PCR, which confirmed that the kanMX marker gene had been excised, leaving behind a single loxP site (FIG. 4). Finally, the Cre expression plasmid was removed from the strains by streaking cells on YPD plates without phleomycin.

### Validation of Genetically Tractable Industrial Yeast Strains

After removal of the kanMX marker, diploids were sporulated and tetrads were dissected. As expected more than 90% of spores were viable. Mating type determination by PCR demonstrated that these spores produced stable heterothallic haploids (FIG. 6). In order to insure that the strains that were created had growth and fermentation characteristics comparable to the initial wild-type strain, growth of resultant haploids was analyzed at 30° C. and 37° C. Fermentation of glucose to ethanol by these cells was also monitored. Both assays did not reveal any substantial difference between the AFY209-216 yeast strains. Finally the haploids were mated and diploid strains AFY217-220 were isolated. AFY219 was selected as representative of the phenotypically similar strains AFY217-220. The AFY219 strain was deposited with the American Type Culture Collection, 10801 University Blvd., Manassas, Va. 20110-2209, USA (ATCC). The deposit of AFY219 was made under the provisions of the Budapest Treaty on the International Recognition of the Deposit of Microorganisms for the Purposes of Patent Procedure (Budapest Treaty). The deposited AFY219 strain was assigned ATCC Accession No. PTA-10443, with a Deposit Date of Oct. 22, 2009.

Thus, genetically tractable industrial yeast strains have been constructed. These strains:
exist as stable haploids of a or α mating type (heterothallic yeast)
contain minimal amounts of exogenous DNA
produce viable spores (close to 100%)
efficiently mate and form viable diploids
form diploids that efficiently sporulate

Importantly, Applicants' industrial yeast strains do not demonstrate abnormalities such as sporulation defects, poor spore viability, polyploidy etc. frequently associated with other industrial yeasts. Moreover, very high germination efficiency combined with robustness of the parental K1-V1116 cells makes them an ideal choice for process development and industrial applications. The successful use of these strains for inactivation of the ethanol biosynthetic pathway is described below.

### Deletion of the ADH1 Gene in the Industrial Yeast Background

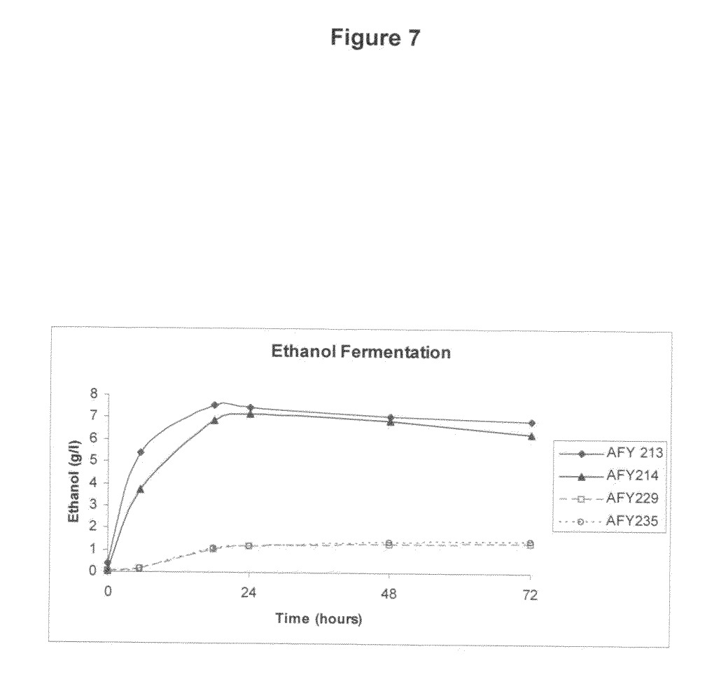

Yeast is a very efficient ethanol producer. To avoid competition between the ethanol biosynthetic pathways and other potentially desired pathways, the former should be blocked by inactivation of gene(s) involved in ethanol production, e.g. ADH1. To delete ADH1 the gene disruption technique described above was used. The gene disruption and verification primers are shown in Table 2. The disruption cassette was amplified using pSH65 plasmid containing the loxP-kanMX-loxP module as a template and used for transformation of the AFY219 heterothallic diploid yeast. Transformation was performed using an electroporation technique (Example 6). G418-resistant colonies were analyzed by diagnostic PCR as described above. Four positive heterozygous diploids were sporulated and tetrads were dissected. Remarkably, in all 4 cases spore viability was 100% demonstrating that genetic selection performed led to dramatic increase of spore viability in comparison with the parental K1-V1116 yeast. Furthermore, segregation of resistance to G418 was 2:2 for each tetrad indicating that there was only one integrated ADH1 disruption cassette per diploid genome. Finally, resistance to G418 was linked to reduced growth rate, a phenotypic abnormality seen in the adh1 mutant cells. When grown in 2% glucose media these strains reached an optical density at 600 nm (OD₆₀₀) of about 0.6 compared to an OD₆₀₀ of about 4.0 for strains without ADH1 gene deletion. As the ADH1 gene is a critical enzyme in the ethanol biosynthetic pathway, the mutated strains also exhibited substantially reduced ethanol fermentation (FIG. 7). Thus, the foregoing industrial yeast strains constructed by Applicants can be used for various manipulations followed by detailed genetic analysis.

All sequence citations, accession numbers, references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Sporulation of the K1-V1116 Diploid Cells

Starvation of diploid yeast cells for nitrogen and carbon sources induces meiosis and spore formation, during which chromosomes replicate and proceed through two divisions to produce haploid nuclei. These nuclei along with surrounding cytoplasm are packed into spores. Four spores forming a tetrad are located in a sick-walled sac (ascus). Sporulation was induced in liquid medium containing 1% CH₃COOK, 0.1% yeast extract and 0.05% glucose (dextrose). This nitrogen-deficient “starvation” medium contains potassium acetate as a carbon source to promote a high level of respiration which induces the diploid yeast strain to sporulate. Yeast cultures were grown in YPD medium overnight and then 100 μL aliquots of resultant cultures were added to 3 mL of sporulation medium. Under these conditions, sporulation was complete within 48 hours at 30° C.

### Example 2

### Preparation and Dissection of Tetrads

To digest the ascus wall, 0.5 mL aliquots of sporulation cultures were centrifuged and resuspended in 50 μL of 10 mM Tris-HCl pH 8.0, 1 M sorbitol. 3 μL of zymolase solution (5 mg/mL in 1 M sorbitol) was added; cell suspensions were incubated for 30 min at 30° C. and then placed on ice. Zymolase-treated spores were streaked in several parallel lines on the surface of a YPD dissection plate. For the analysis of yeast tetrads a standard light microscope with a stage that is movable along both X and Y axes was used. The microscope was modified with an assembly for mounting an inverted Petri dish and a micromanipulator for holding a fine glass needle. Spores from the same tetrad were separated using the needle and placed in a line at 5 mm intervals. Normally thirteen tetrads were dissected on a plate. Dissection plates were incubated for 2 days at 30° C. and used for further analysis.

### Example 3

### Gene Disruption Using the loxP-kanMX-loxP Disruption Cassettes

The chromosomal HO and ADH1 genes were inactivated by PCR-based gene deletion using the pUG6 plasmid (Gueldener et al., 1996) as a PCR template to create a DNA fragment that directed replacement of the chromosomal ORFs with the kanMX gene by homologous recombination in diploid yeast cells. Two cassettes were amplified using HO and ADH1 disruption primers (Table 2). The 5′-50 nucleotides of the primers were designed to be homologous to target gene sequences upstream of the ATG start codon or downstream of the termination codon. The 3′-segments were designed to be homologous to sequences to the right and to the left of loxP motifs of the disruption cassettes (FIG. 3).

To confirm correct integration of the disruption cassettes into the HO and ADH1 loci, diagnostic PCR was performed on the G418 resistant transformants using a combination of corresponding target gene-specific primers (A, D) and disruption cassette specific primers (B, C) (Table 2). The heterozygous diploids were sporulated, and tetrads were dissected as described in Example 2.

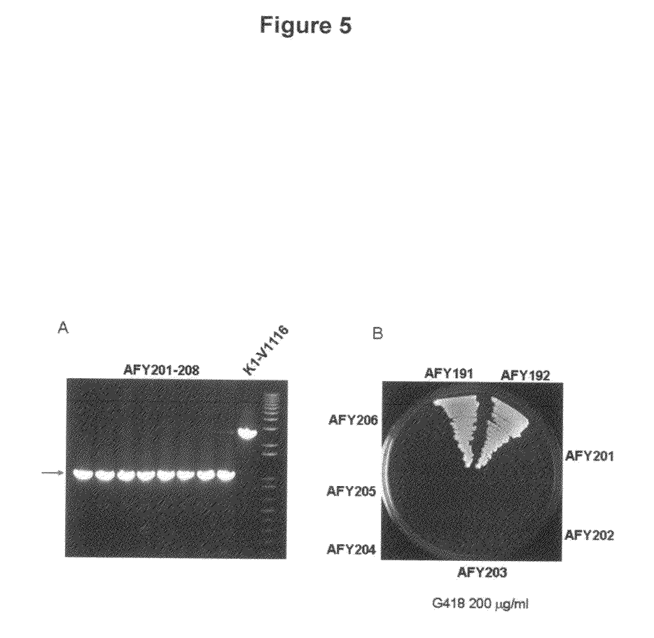

To use the kanMX marker repeatedly for several gene disruptions in one strain, it is necessary to eliminate this antibiotic resistance gene from the successfully disrupted genome. The ho and adh1 mutant strains, in which corresponding genes were disrupted by the loxP-kanMX-loxP cassettes, were transformed with the Cre recombinase expression plasmid pSH65 that carries the ble marker gene and the cre gene under the control of the inducible GAL1 promoter (Guldener at al., 2002) (FIG. 4). Expression of the Cre recombinase was induced by shifting cells from glucose to galactose medium and incubating for 2 hours in the galactose containing medium. Cells that lost the kanMX marker gene were detected by replica plating yeast colonies on YPD plates containing 200 μg/mL G418. Loss of the kanMX marker gene was further verified by diagnostic PCR (FIG. 5). The Cre expression plasmid was removed from these strains by growing cells overnight in YPD medium without phleomycin. Without the selection pressure created by the phleomycin antibiotic, the yeast spontaneously loses the now unnecessary plasmid which had previously provided resistance to the antibiotic.

### Example 4

### Determination of the Mating Type by PCR

Yeast cells can exist as haploids of a or α mating type or a/α diploids. Genetic analysis often involves determination of the mating type by crossing the strain in question with strains of known mating type. Such analysis is slow and requires specific auxotrophic markers. Given that industrial strains are normally prototrophs, an alternative method based on PCR was used for mating type determination. Specifically, the PCR assay determines the DNA sequence at the mating type locus thus identifying the mating type of the haploid. Also, it positively identifies diploids (Huxley et al., 1990). Three oligonucleotide primers were used in this assay. MATtest corresponds to a sequence at the right of the MAT locus and directed towards it. MATalpha corresponds to a sequence within the α-specific DNA located at MATα and HMLα loci. MATa corresponds to a sequence within a-specific DNA located at MATa and HMRa. When these primers are used in a single PCR, DNAs at MATa and MATα loci generated 0.54 kb and 0.4 kb products, respectively (FIG. 6).

The PCR assay was performed using yeast colonies that were grown for 2 days. Roughly 5-10% of a single colony was incubated in 2.5 μL of 0.02 N NaOH for 10 min at 96° C. and then 25 μL of the PCR primer mixture was added to the cell suspension. 30 PCR cycles (96° C. for 30 sec, 50° C. for 30 sec and 68° C. for 1 min) were performed using Platinum High Fidelity Taq Polymerase (Invitrogen, Carlsbad, Calif.).

### Example 5

### Diploid Construction

Diploids were constructed by mating strains of opposite mating type on the surface of YPD plates. Cells from freshly grown colonies of each haploid parent were mixed with wood applicators in a circle ˜0.5 mm and incubated for 4 hours at 30° C. The mating mixtures then were streaked onto another YPD plate for diploid selection. Diploids were isolated by physically pulling zygotes from the mating mixture using a dissection microscope. Zygotes can be easily identified by their characteristic shape (Ausubel et al., 2002).

### Example 6

### Transformation of the Yeast Cells by Electroporation

Chemical transformation using lithium acetate is the most common approach for introduction of DNA into laboratory yeast strains. However, this technique is not suitable for industrial strains. On the other hand, electroporation was shown to be effective for at least several industrial strains. (Ho et al., 1998; Walker, 1998; Husnik et al., 2006; Olivera et al., 2007). This technique is reasonably fast and provides satisfactory transformation efficiency. To prepare electrocompetent cells, 200 mL of YPD was inoculated with 5 mL of yeast overnight culture. Cells were grown for 5 hours to OD₆₀₀ ˜1-1.5 and then pelleted by centrifugation at 4000 g for 5 min at 4° C. Cells were resuspended in 10 mL of TE buffer containing 0.1 M Lithium Acetate and incubated for 45 min at 30° C. 0.25 mL of 1 M DTT was added to cell suspension and incubation continued for another 15 minutes. Cells were centrifuged, washed twice with 50 mL of ice-cold water and once with 25 mL of ice-cold 1 M sorbitol. The cell pellet was resuspended in 200 mL of 1 M sorbitol, and the suspension was kept on ice and used as soon as possible for electroporation.

For each sample to be electroporated, a sterile tube with 40 mL of electrocompetent cell suspension and a 0.2 cm cuvette were prepared and placed on ice. DNA to be transformed into the cells was added to the cell suspension; the resulting mixture was incubated on ice for 5 min and then transferred to the cuvette. Electroporation was performed at 1.5 kV and 1 mL of 1 M sorbitol was immediately added to the cuvette. For G418 selection, samples were transferred to 15 mL sterile tubes, 5 mL of YPD containing 1 M sorbitol were added to each tube and the resultant suspensions were incubated for 2 hours at 30° C. Aliquots of the electroporated cells were plated on YPD plates containing antibiotic and incubated at 30° C. for 2 days.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

TABLE 1
Yeast strains

| | |
|---|---|
| AFY133 | K1-V1116 |
| AFY150 | product of the AFY133 dissection, viable spore 1d |
| AFY151 | product of the AFY133 dissection, viable spore 3d |
| AFY154 | product of the AFY133 dissection, viable spore 11b |
| AFY157 | product of the AFY150 dissection, spore 1b |
| AFY159 | product of the AFY150 dissection, spore 1d |
| AFY191-200 | a/α ho::kanMX/ho::kanMX |
| AFY201-208 | a/α ho/ho |
| AFY209-216 | a or α ho |
| AFY217-220 | a/α ho/ho |
| AFY225 | AFY219 derivative, α adh1::kanMX |
| AFY226 | AFY219 derivative, a adh1::kanMX |
| AFY229 | AFY225 derivative, α adh1 |
| AFY235 | AFY226 derivative, a adh1 |

TABLE 2
List of oligonucleotides

Gene disruption primers
HO
TCTAAATCCATATCCTCATAAGCAGCAATCAATTCTATCTATACTTTAAA
cagctgaagcttcgtacgc
(SEQ ID NO: 1)
ATTAAATTTTACTTTTATTACATACAACTTTTTAAACTAATATACACATT
gcataggccactagtggatctg
(SEQ ID NO: 2)
ADH1
GCACAATATTTCAAGCTATACCAAGCATACAATCAACTATCTCATATACA
cagctgaagcttcgtacgc
(SEQ ID NO: 3)
TTTTTTATAACTTATTTAATAATAAAAATCATAAATCATAAGAAATTCGC
gcataggccactagtggatctg
(SEQ ID NO: 4)
Verification primers/target gene-specific

| | |
|---|---|
| HOA | CGCAAGTCCTGTTTCTATGCC |
| | (SEQ ID NO: 5) |
| HOD | TTCCAAGTCCAAGATTGAAGCTG |
| | (SEQ ID NO: 6) |
| ADH1A | TCTCTCTCCCCCGTTGTTGT |
| | (SEQ ID NO: 7) |
| ADH1D | CTCAGGTAAGGGGCTAGTAG |
| | (SEQ ID NO: 8) |

Verification primers/disruption cassette specific

| | |
|---|---|
| kan-B | GGATGTATGGGCTAAATG |
| | (SEQ ID NO: 9) |
| kan-C | CCTCGACATCATCTGCCC |
| | (SEQ ID NO: 10) |

Mating type determination primers

| | |
|---|---|
| MATtest | AGTCACATCAAGATCGTTTATGG |
| | (SEQ ID NO: 11) |
| MATalpha | GCACGGAATATGGGACTACTTCG |
| | (SEQ ID NO: 12) |
| MATa | ACTCCACTTCAAGTAAGAGTTTG |
| | (SEQ ID NO: 13) |

### REFERENCES

Ausubel F M, Brent R, Kingston R E, More D D, Seidman J G, Smith J A, Struhl. 2002. Short Protocols in Molecular Biology. John Wiley and Sons. New York. NY, 2, unit 13.2.
Borneman A. R., Forgan A. H., Pretorius I. S., Chambers P. J. 2008 Comparative genome analysis of a Saccharomyces cerevisiae wine strain. FEMS Yeast Res. 8. 1185-1195.
Herskowitz I. 1988. Life cycle of the budding yeast Saccharomyces cerevisiae. Microbiol. Rev. 52.536-553.
Ho N. W. Y., Chen Z., Brainard A. P. 1998. Genetically engineered Saccharomyces yeast capable of cofermentation of glucose and zylose. Appl. Env. Microbiol. 64. 1852-1859
Husnik J., Volschek H., Bauer J. Colavizza D., Luo Z., van Vuuren H. J. J. 2006. Metabolic engineering of malolactic wine yeast. Metabol. Eng. 8.315-323.
Huxley C., Green E. D., Dunham I. 1990. Rapid assessment of S. cerevisiae mating type by PCR. Trends Genet. 6. 236.
Güldener U., Heck S., Fielder T., Beinhauer J., Hegemann J. H. 1996. A new efficient gene disruption cassette for repeated use in budding yeast. Nucleic Acids Res. 13. 2519-2524.
Gueldener U., Heinisch J., Koehler G. J., Voss D., Hegemann J. H. 2002. A second set of loxP marker cassettes for Cre-mediated multiple gene knockouts in budding yeast. Nucleic Acids Res. 30. e23.
Johnston J. R., Baccary C., Mortimer R. K. Genotypic characterization of strains of commercial wine yeast by tetrad analysis. 2000. Res. Microbiol. 151.583-590.
Kelsall D. R., Lyons T. P. Practical management of yeast: conversion of sugars to ethanol. 2003. In the Alcohol Textbook. K. A. Jacques, D. R. Kelsall editors. Nottingham University Press, Nottingham. 121-134.
Kielland-Brandt M. Industrial Saccharomyces Yeasts. 1994. In Molecular Genetics of Yeast J. R. Johnston editor. Oxford University Press, Oxford, UK. 247-250.
Nasmyth K. A. 1982. Molecular genetics of yeast mating type. Ann. Rev. Genet. 16.439-500.
Nickoloff J. A., Chen E. Y., Heffron F. 1986. A 24-base-pair DNA sequence from the MAT locus stimulates intergenic recombination in yeast. Proc. Natl. Acad. Sci. USA. 83. 7831-7835.
Olivera C., Teixeira J. A., Lima N., Da Silva N. A., Domingues L. 2007. Development of stable flocculent Saccharomyces cerevisiae strain for continuous Aspergillis niger b-galactosidase production. J. Biosci. Bioeng. 103.318-324.
Ooi B. G., Lankford K. R. 2009. Strategy for adopting wine yeast for bioethanol production. Int. J. Mol. Sci. 10.385-394.
Strathern J. N., Klar A. J., Hicks J. B., Abraham J. A., Ivy J. M., Nasmyth K. A., McGill C. Homothallic switching of yeast mating type cassettes is initiated by a double-stranded cut in the MAT locus. 1982. Cell. 31.183-192.
Walker G. M. 1998. Yeast Physiology and Biotechnology. John Wiley and Sons. New York. NY, unit 4.4.2.3.

What is claimed is:

1. A yeast strain, comprising: an industrial yeast strain wherein said yeast strain sporulates efficiently and upon sporulation yields at least 90% viable spores.

2. The yeast strain of claim 1, wherein the strain upon sporulation yields about 100% viable spores.

3. The yeast strain of claim 1, wherein said yeast is Saccharomyces cerevisiae.

4. The yeast strain of claim 3, wherein the Saccharomyces cerevisiae strain is derived from a K1-V1116 strain.

5. The yeast strain of claim 4, wherein at least one copy of the HO gene has been deleted.

6. The yeast strain of claim 5, wherein the yeast is a stable heterothallic haploid.

7. The yeast strain of claim 6, wherein the heterothallic haploid has the a mating type.

8. The yeast strain of claim 6, wherein the heterothallic haploid has the α mating type.

9. The yeast strain of claim 6, wherein an ethanol biosynthetic pathway has been inactivated.

10. The yeast strain of claim 9, wherein an ethanol biosynthetic pathway has been inactivated by deleting the ADH1 gene.

11. The yeast strain of claim 6, wherein the strain has been further modified by recombinant DNA technology.

12. The yeast strain of claim 5, wherein the yeast is a heterothallic diploid.

13. The yeast strain of claim 12, wherein an ethanol biosynthetic pathway has been inactivated.

14. The yeast strain of claim 13, wherein an ethanol biosynthetic pathway has been inactivated by deleting the ADH1 gene.

15. A yeast strain selected from the group consisting of a yeast deposited under ATCC Accession No. PTA-10443, a yeast phenotypically similar to the yeast deposited under ATCC Accession No. PTA-10443, progeny thereof and derivatives thereof.

16. The yeast strain of claim 15, wherein said yeast strain is a diploid strain obtained by mitosis.

17. The yeast strain of claim 15, wherein said yeast strain is a haploid strain obtained by sporulation.

18. A method of generating a genetically tractable yeast strain, comprising:
(a) in a population of industrial yeast cells, inducing the yeast to sporulate; and
(b) selecting viable spores from those yeast cells that sporulate efficiently.

19. The method of claim 18, further comprising the step of:
(c) deleting at least one copy of the HO gene from the selected yeast;
(d) inducing the yeast to sporulate;
(e) identifying and selecting spores with the deleted HO gene,
thereby producing stable heterothallic haploid yeast.

20. The method of claim 19, further comprising the step of:
(f) mating heterothallic haploid yeast of opposite mating types
thereby producing heterothallic diploid yeast.

21. The method of claim 20, wherein said steps (a) and (b) are repeated two or more times.

22. The method of claim 21, wherein inducing yeast to sporulate comprises starvation of said yeast.

23. The method of claim 22, wherein said yeast is Saccharomyces cerevisiae.

24. The method of claim 23, wherein the Saccharomyces cerevisiae is derived from a K1-V1116.

25. A yeast strain produced by the method of claim 18.

26. A method of producing a fermentation product which comprises fermenting a carbon substrate with the yeast strain of claim 1 under conditions where said fermentation product is produced.

27. The method of claim 26 further comprising the step of recovering said fermentation product.

28. A method of producing a fermentation product which comprises fermenting a carbon substrate with the yeast strain of claim 15 under conditions where said fermentation product is produced.

29. The method of claim 28 further comprising the step of recovering said fermentation product.

30. A method of producing a fermentation product which comprises fermenting a carbon substrate with the yeast strain produced by the method of claim 18 under conditions where said fermentation product is produced.

31. The method of claim 30 further comprising the step of recovering said fermentation product.

32. A method of manufacturing a yeast strain which comprises culturing the yeast strain of claim 1 in a medium under conditions where said yeast strain grows.

33. A method of manufacturing a yeast strain which comprises culturing the yeast strain of claim 15 in a medium under conditions where said yeast strain grows.

34. A method of manufacturing a yeast strain which comprises culturing the yeast strain produced by the method of claim 18 in a medium under conditions where said yeast strain grows.
